(19) United States
(12) Patent Application Publication
(10) Pub. No.: US 2024/0252313 A1
(43) Pub. Date: Aug. 1, 2024

(54) COMPOSITE SKIRTS FOR PROSTHETIC VALVE DEVICES

(71) Applicant: Medtronic, Inc., Minneapolis, MN (US)

(72) Inventors: Yogesh DAREKAR, Irvine, CA (US); Brenda MCINTIRE, Walnut Creek, CA (US); Eric PIERCE, Mission Viejo, CA (US)

(21) Appl. No.: 18/561,055

(22) PCT Filed: May 12, 2022
(86) PCT No.: PCT/IB2022/054438

§ 371 Date: Nov. 15, 2023

Related U.S. Application Data
(60) Provisional application No. 63/193,650, filed on May 27, 2021.

(51) Int. Cl. A61F 2/24

(52) U.S. Cl. CPC A61F 2/2418; A61F 2210/0076; A61F 2230/0017; A61F 2250/0018

(57) ABSTRACT

Composite skirts for a stent or frame of a valve prosthesis are configured to prevent billowing of the skirt material that spans across the side openings of the frame of the valve prosthesis, as such billowing may undesirably result in contact between the skirt and leaflets of the valve prosthesis after the valve prosthesis is deployed in situ. In a first embodiment, a composite skirt is formed by alternating first and second segments of a first stiffness and a second stiffness, respectively, that alternate in a circumferential direction. The second stiffness is higher than the first stiffness. Each segment of the alternating first and second segments extends in an axial direction for substantially an entire length of the composite skirt. In a second embodiment, a multi-layer skirt is formed by a porous fabric material and a thin polymeric coating covering the fabric material.

### FIELD

The present technology is generally related to prosthetic valve devices, and in particular is directed to prosthetic valve devices including a skirt.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and shut in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

The present disclosure relates to improvements in a heart valve prosthesis to ensure that the heart valve prosthesis has a low profile for transcatheter delivery through a patient's vasculature.

### SUMMARY

According to a first embodiment hereof, the present disclosure provides a prosthesis having a radially expanded configuration and a radially compressed configuration. The prosthesis includes a frame including a plurality of crowns and a plurality of struts with each crown being formed between a pair of opposing struts. A plurality of side openings is defined by edges of the plurality of crowns and the plurality of struts. The prosthesis also includes a skirt coupled to a surface of the frame. The composite skirt extends over at least one side opening of the plurality of side openings of the frame. The composite skirt is formed by alternating first and second segments of a first stiffness and a second stiffness, respectively, that alternate in a circumferential direction. The second stiffness is higher than the first stiffness. Each segment of the alternating first and second segments extend in an axial direction for substantially an entire length of the composite skirt.

In an aspect of the first embodiment, and in combination with any other aspects herein, the disclosure provides that the first segments of the composite skirt are configured to fold radially inward when the prosthesis is in the radially compressed configuration such that the composite skirt has a pleated configuration when the prosthesis is in the radially compressed configuration.

In an aspect of the first embodiment, and in combination with any other aspects herein, the disclosure provides that the second segments of the composite skirt are configured to prevent billowing of the composite skirt when the prosthesis is in the radially expanded configuration.

In an aspect of the first embodiment, and in combination with any other aspects herein, the disclosure provides that the alternating first and second segments include between five and twenty first segments and between five and twenty second segments.

In an aspect of the first embodiment, and in combination with any other aspects herein, the disclosure provides that the each of the first and second segments are the same size.

In an aspect of the first embodiment, and in combination with any other aspects herein, the disclosure provides that the second stiffness is at least two times greater than the first stiffness.

In an aspect of the first embodiment, and in combination with any other aspects herein, the disclosure provides that at least one opening of the plurality of side openings is substantially diamond-shaped and is defined by a total of four struts.

In an aspect of the first embodiment, and in combination with any other aspects herein, the disclosure provides that each opening of the plurality of side openings is substantially diamond-shaped and is defined by a total of four struts.

In an aspect of the first embodiment, and in combination with any other aspects herein, the disclosure provides that a perimeter of the frame includes a row of side openings, the row including between six side openings and nine side openings.

In an aspect of the first embodiment, and in combination with any other aspects herein, the disclosure provides that the composite skirt is coupled to an inner surface and the composite skirt extends over each opening of the plurality of side openings of the frame.

In an aspect of the first embodiment, and in combination with any other aspects herein, the disclosure provides that the frame is formed from a self-expanding material.

In an aspect of the first embodiment, and in combination with any other aspects herein, the disclosure provides that the frame is an inner frame and the prosthesis further comprises an outer frame coupled to the inner frame, the outer frame radially surrounding the inner frame.

In an aspect of the first embodiment, and in combination with any other aspects herein, the disclosure provides that the prosthesis is a heart valve prosthesis and the prosthesis further comprises a prosthetic valve component disposed within and secured to the frame, the prosthetic valve being configured to block blood flow in one direction to regulate blood flow through a central lumen of the frame. In an embodiment, the heart valve prosthesis is configured for placement within a mitral heart valve or a tricuspid heart valve in situ.

According to a second embodiment hereof, the present disclosure provides a prosthesis having a radially expanded configuration and a radially compressed configuration. The prosthesis includes a frame including a plurality of crowns and a plurality of struts with each crown being formed between a pair of opposing struts. A plurality of side openings is defined by edges of the plurality of crowns and the plurality of struts. The prosthesis also includes a multi-layer skirt coupled to a surface of the frame. The multi-layer skirt extends over at least one side opening of the plurality of side openings of the frame. The multi-layer skirt is formed by a fabric material and a polymeric coating covering the fabric material such that the polymeric coating defines an inner circumferential surface of the multi-layer skirt. The fabric material has a plurality of fibers with a porosity less than 5%. The polymeric coating has a thickness between 2 and 10 um.

In an aspect of the second embodiment, and in combination with any other aspects herein, the disclosure provides that the polymeric coating is formed from polyurethane, polycarbonate urethane, or polytetrafluoroethylene.

In an aspect of the second embodiment, and in combination with any other aspects herein, the disclosure provides that the polymeric coating is configured to prevent billowing of the multi-layer skirt when the prosthesis is in the radially expanded configuration.

In an aspect of the second embodiment, and in combination with any other aspects herein, the disclosure provides that at least one opening of the plurality of side openings is substantially diamond-shaped and is defined by a total of four struts.

In an aspect of the second embodiment, and in combination with any other aspects herein, the disclosure provides that each opening of the plurality of side openings is substantially diamond-shaped and is defined by a total of four struts.

In an aspect of the second embodiment, and in combination with any other aspects herein, the disclosure provides that a perimeter of the frame includes a row of side openings, the row including between six side openings and nine side openings.

In an aspect of the second embodiment, and in combination with any other aspects herein, the disclosure provides that the multi-layer skirt is coupled to an inner surface or an outer surface of the frame and the multi-layer skirt extends over each opening of the plurality of side openings of the frame.

In an aspect of the second embodiment, and in combination with any other aspects herein, the disclosure provides that the frame is formed from a self-expanding material.

In an aspect of the second embodiment, and in combination with any other aspects herein, the disclosure provides that the frame is an inner frame and the prosthesis further comprises an outer frame coupled to the inner frame, the outer frame radially surrounding the inner frame.

In an aspect of the second embodiment, and in combination with any other aspects herein, the disclosure provides that the prosthesis is a heart valve prosthesis and the prosthesis further comprises a prosthetic valve component disposed within and secured to the frame, the prosthetic valve being configured to block blood flow in one direction to regulate blood flow through a central lumen of the frame. In an embodiment, the heart valve prosthesis is configured for placement within a mitral heart valve or a tricuspid heart valve in situ.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 depicts a perspective view of a transcatheter valve prosthesis in accordance with an aspect of the disclosure.

FIG. 2 depicts a perspective view of a valve support of the transcatheter valve prosthesis of FIG. 1 with a prosthetic valve component secured therein in accordance with an aspect of the disclosure.

FIG. 3 depicts an atrial end view of the transcatheter valve prosthesis shown in FIG. 1 in accordance with an aspect of the disclosure.

FIG. 4 depicts a ventricular end view of the transcatheter valve prosthesis shown in FIG. 1 in accordance with an aspect of the disclosure.

FIG. 5 is an enlarged side view of a side opening of the valve support of FIG. 2.

FIG. 6A is a perspective ventricular end view of the transcatheter valve prosthesis shown in FIG. 1 in accordance with an aspect of the disclosure.

FIG. 6B is a ventricular end view of the transcatheter valve prosthesis shown in FIG. 1 in accordance with an aspect of the disclosure.

FIG. 7A is a side view of the valve support of the transcatheter heart valve prosthesis of FIG. 1, wherein the valve support includes a composite skirt in accordance with an aspect of the disclosure and wherein the valve support is in its radially expanded configuration.

FIG. 7B is a perspective view of the valve support of FIG. 7A.

FIG. 8 depicts the composite skirt of FIG. 7A removed from the valve support and laid flat for illustrative purposes only.

FIG. 9 is a perspective view of the valve support of FIG. 7A, wherein the valve support is in its radially compressed configuration.

FIG. 10 is a perspective view of the valve support of FIG. 7A, wherein the valve support is in its radially expanded configuration.

FIG. 11A is a side view of the valve support of the transcatheter heart valve prosthesis of FIG. 1, wherein the valve support includes a multi-layer skirt in accordance with another aspect of the disclosure and wherein the valve support is in its radially expanded configuration.

FIG. 11B is a perspective view of the valve support of FIG. 11A.

FIG. 12 depicts a top view of the multi-layer skirt of FIG. 11A removed from the valve support for illustrative purposes only.

### DETAILED DESCRIPTION

Specific embodiments of the present invention are now described with reference to the figures, wherein like reference numbers indicate identical or functionally similar elements. The terms “distal” and “proximal”, when used in the following description to refer to a native vessel, native valve, or a device to be implanted into a native vessel or native valve, such as a transcatheter valve prosthesis, are with reference to the direction of blood flow. Thus, “distal” and “distally” refer to positions in a downstream direction with respect to the direction of blood flow and the terms “proximal” and “proximally” refer to positions in an upstream direction with respect to the direction of blood flow.

Embodiments hereof relate to a composite skirt having long-lasting durability and superior implant performance for a stent or frame of a valve prosthesis. As will be explained in more detail herein, the composite skirt is configured to prevent billowing of the skirt material that spans across the side openings of the frame of the valve prosthesis, as such billowing may undesirably result in contact between the skirt and the leaflets of the valve prosthesis after the valve prosthesis is deployed in situ. If the leaflets of the valve prosthesis contact the skirt during opening and closing in situ, such contact may cause early leaflet tissue abrasion as well as early skirt abrasion due to the undesired billowing of the skirt. Additionally, the greater relative motion between the skirt and the frame may further induce early skirt abrasion. Early leaflet tissue abrasion and/or early skirt abrasion has a negative impact on the long-term durability of the valve prosthesis. The composite skirt disclosed herein is configured to limit the radial motion or billowing of the skirt material, thereby minimizing risk of damage to both the skirt and the leaflets.

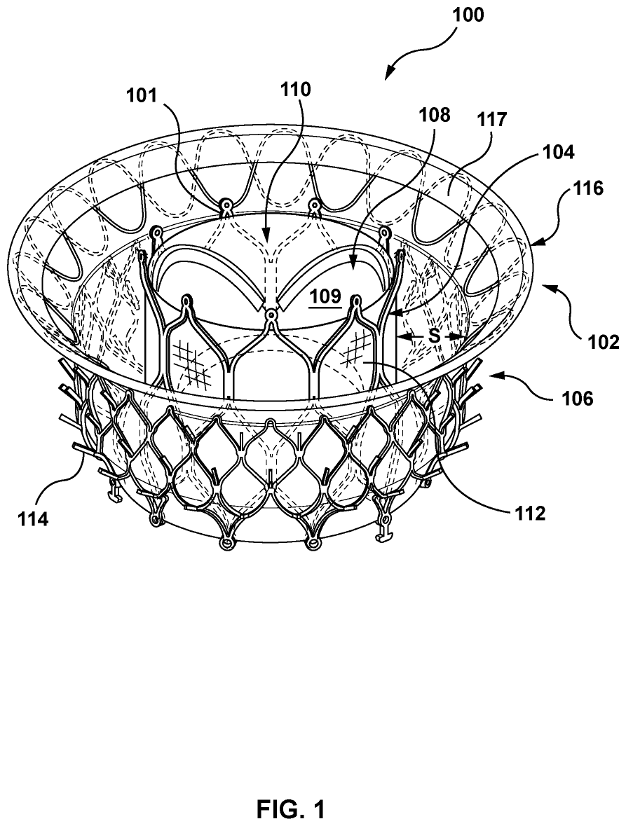

FIGS. 1-6 illustrate a transcatheter heart valve prosthesis 100 that may be utilized with embodiments of composite skirts described herein. The heart valve prosthesis 100 is illustrated herein in order to facilitate description of the present invention. The following description of the transcatheter heart valve prosthesis 100 is merely exemplary in nature and is not intended to limit the invention or the application and uses of the invention. It is understood that any number of alternate heart valve prostheses can be used with the composite skirts described herein. Other non-limiting examples of transcatheter heart valve prostheses that can be used with the composite skirts described herein are described in U.S. application Ser. No. 16/853,851 to McVeigh et al., U.S. Pat. No. 9,034,032 to McLean et al. and International Patent Application No. PCT/US2014/029549 to McLean et al, U.S. Patent Application Publication No. 2012/0101572 to Kovalsky et al., U.S. Patent Application Publication No. 2012/0035722 to Tuval, U.S. Patent Application Publication No. 2006/0265056 to Nguyen et al., U.S. Patent Application Publication No. 2007/05409266 to Birdsall, and U.S. Patent Application Publication No. 2007/05409269 to Dolan et al., each of which is incorporated by reference herein in its entirety. Although the transcatheter heart valve prosthesis 100 is a heart valve prosthesis configured for placement within a mitral or tricuspid heart valve, embodiments of composite skirts described herein may be utilized with any valve prosthesis having a skirt. For example, embodiments of composite skirts described herein may be utilized with a transcatheter heart valve configured for placement within a pulmonary; aortic, mitral, or tricuspid valve, or may be utilized with a transcatheter valve prosthesis configured for placement within a venous valve or within other body passageways where it is deemed useful. There is no intention to be bound by any expressed or implied theory presented in the preceding technical field, background, brief summary or the following detailed description. In addition, embodiments of composite skirts described herein may be utilized with any stent or frame having a skirt for which it is desirable to limit billowing, and it is not required that the stent or frame include a prosthetic valve component disposed therein.

A perspective view of the transcatheter heart valve prosthesis 100 in accordance with an aspect of the disclosure is shown in FIG. 1. The transcatheter heart valve prosthesis 100 is configured to be radially compressed into a reduced-diameter crimped configuration for delivery within a vasculature (not shown) and to return to an expanded, deployed configuration, as shown in FIG. 1. Stated another way, the transcatheter heart valve prosthesis 100 has a crimped configuration for delivery within a vasculature and an expanded configuration for deployment within a native heart valve. In accordance with embodiments hereof, when in the crimped configuration, the transcatheter heart valve prosthesis 100 has a low profile suitable for delivery to and deployment within a native heart valve via a suitable delivery catheter that may be tracked to the deployment site of the native heart valve of a heart via any one of a transseptal, retrograde, or transapical approach. The transcatheter heart valve prosthesis 100 includes a stent or frame 102 and a prosthetic valve component 108 including at least one leaflet disposed within and secured to the frame 102.

Any portion of the frame 102 described herein as an element of a heart valve prosthesis 100 may be made from any number of suitable biocompatible materials, e.g., stainless steel, nickel titanium alloys such as Nitinol™, cobalt chromium alloys such as MP35N, other alloys such as ELGILOY® (Elgin, Ill.), various polymers, pyrolytic carbon, silicone, polytetrafluoroethylene (PTFE), or any number of other materials or combination of materials. A suitable biocompatible material would be selected to provide the transcatheter heart valve prosthesis 100 to be configured to be compressed into a reduced-diameter crimped configuration for transcatheter delivery to a native valve, whereby release from a delivery catheter returns the prosthesis to an expanded, deployed configuration.

In an aspect of the disclosure, the frame 102 of the transcatheter heart valve prosthesis 100 includes a valve support 104 at least partially surrounded by and coupled to an anchor element 106. The valve support 104 is a tubular stent-like or frame structure that defines a central lumen 110 from an inflow end 101 of the valve support 104 to an outflow end 103 of the valve support 104. The valve support 104 is configured to support the prosthetic valve component 108 therein, which will be described in more detail below. In an embodiment, the valve support 104 has a substantially cylindrical shape in which the outflow end 103 of the valve support 104 has a diameter that is substantially the same as a diameter of the inflow end 101 of the valve support 104.

The valve support 104 includes a skirt 112 coupled to a surface thereof. More particularly, the skirt 112 is coupled to an inner surface of the valve support 104 to line a portion thereof. Alternatively, the skirt 112 may be coupled to an outer surface of the valve support 104 to enclose a portion thereof as would be known to one of ordinary skill in the art of prosthetic valve construction. The skirt 112 may be a natural or biological material such as pericardium or another membranous tissue such as intestinal submucosa. Alternatively, the skirt 112 may be a low-porosity woven fabric, such as polyester, Dacron fabric, or PTFE, which creates a one-way fluid passage when attached to the stent. In one embodiment, the skirt 112 may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. These and other appropriate cardiovascular fabrics are commercially available from Bard Peripheral Vascular, Inc. of Tempe, Ariz., for example.

In an aspect of the disclosure, the anchor element 106 is a stent-like or frame structure that functions as an anchor for the transcatheter heart valve prosthesis 100 to secure its deployed position within a native annulus. The anchor element 106 is a substantially cylindrically-shaped structure that is configured to engage heart tissue at or below an annulus of a native heart valve, such as an annulus of a native mitral valve or native tricuspid valve. At the inflow end 101 of the valve support 104, the anchor element 106 is radially spaced a distance S from the valve support 104 to mechanically isolate the inflow end 101 of the valve support 104 from the anchor element 106. The anchor element 106 includes one or more cleats or prongs 114 that extend outward from an exterior side thereof to engage heart tissue. In another embodiment, the anchor element 106 may employ barbs, spikes, or other tissue fixation mechanisms for engaging heart tissue.

The transcatheter heart valve prosthesis 100 further includes a brim or rim element 116 that extends outwardly from an upstream end of the anchor element 106. The brim element 116 includes overlapping, 180 degree out of phase sinusoidal wire forms that are attached and hinged to the anchor element 106 by a suitable biocompatible low-profile fabric 117 used in bioprosthetic implants namely endovascular grafts, heart valves or left atrial appendage devices to promote bio-integration, such as woven polyethylene terephthalate (PET) fabric. The brim element 116 may act as an atrial retainer, if present, and to serve such a function the brim element 116 may be configured to engage tissue above a native annulus, such as a supra-annular surface or some other tissue in the left atrium, to thereby inhibit downstream migration of a prosthetic heart valve 100, for e.g., during atrial systole.

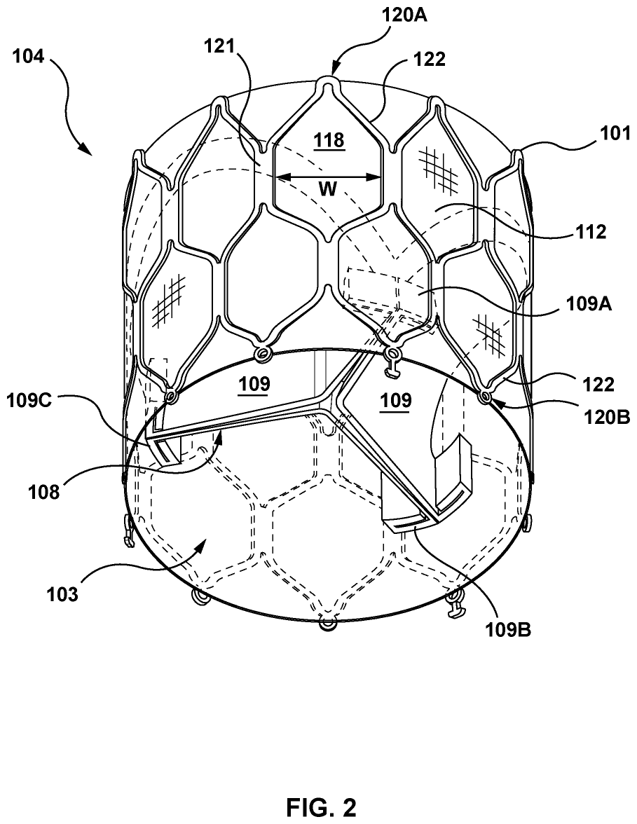

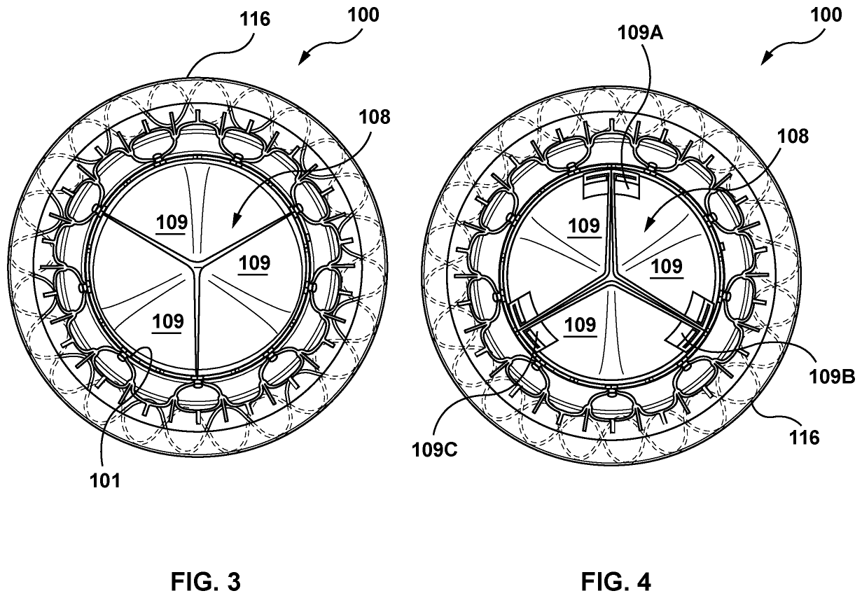

The prosthetic valve component 108 of the transcatheter heart valve prosthesis 100 is capable of regulating flow therethrough via valve leaflets that may form a replacement valve. FIGS. 1-6 illustrate an exemplary prosthetic valve component having three leaflets, although a single leaflet or bicuspid leaflet configuration may alternatively be used in embodiments hereof. When deployed in situ, the prosthetic valve component 108 in a closed state is configured to block blood flow in one direction to regulate blood flow through the central lumen 110 of the valve support 104. FIG. 2 depicts a perspective view of the valve support 104 with a prosthetic valve component 108 secured therein, the valve support 104 being shown in FIG. 2 removed from the remainder of the transcatheter heart valve prosthesis 100 shown in FIG. 1 for ease of illustration. FIG. 3 depicts an atrial or inflow end view of the transcatheter heart valve prosthesis 100 shown in FIG. 1, and FIG. 4 depicts a ventricular or outflow end view of the transcatheter heart valve prosthesis 100 shown in FIG. 1. The prosthetic valve component 108 includes valve leaflets 109, e.g., three valve leaflets 109, that are disposed to coapt within an upstream portion of the valve support 104 with leaflet commissures 109A, 109B, 109C of the valve leaflets 109 being secured within a downstream portion of the valve support 104, such that the valve leaflets 109 open during diastole. Leaflets 109 are attached along their bases to the valve support 104, for example, using sutures or a suitable biocompatible adhesive. Adjoining pairs of leaflets 109 are attached to one another at their lateral ends to form leaflet commissures 109A, 109B, 109C. The orientation of the leaflets 109 within the valve support 104 depends upon on which end of the transcatheter heart valve prosthesis 100 is the inflow end and which end of the transcatheter heart valve prosthesis 100 is the outflow end, thereby ensuring one-way flow of blood through the transcatheter heart valve prosthesis 100.

The valve leaflets 109 may be attached to the skirt 112. The valve leaflets 109 may be formed of various flexible materials including, but not limited to natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to stretching, deforming, or fatigue.

For delivery, the transcatheter heart valve prosthesis 100 is radially compressed into a reduced-diameter crimped configuration onto a delivery system for delivery within a vasculature. As known in the art, the delivery system includes an inner shaft that receives the transcatheter heart valve prosthesis 100 on a distal portion thereof and an outer sheath or capsule that is configured to compressively retain the transcatheter heart valve prosthesis 100 on the distal portion of the inner shaft during delivery. Stated another way, the outer sheath or capsule surrounds and constrains the transcatheter heart valve prosthesis 100 in the radially compressed or crimped configuration. An exemplary delivery system for delivering the transcatheter heart valve prosthesis 100 is described in U.S. Pat. No. 9,034,032 to McLean et al. and International Patent Application No. PCT/US2014/029549 to McLean et al, previously incorporated by reference herein. However, it will be apparent to one of ordinary skill in the art that other delivery systems may be utilized and that the components of the delivery system may vary depending upon the configuration and structure of the transcatheter valve prosthesis that is being delivered.

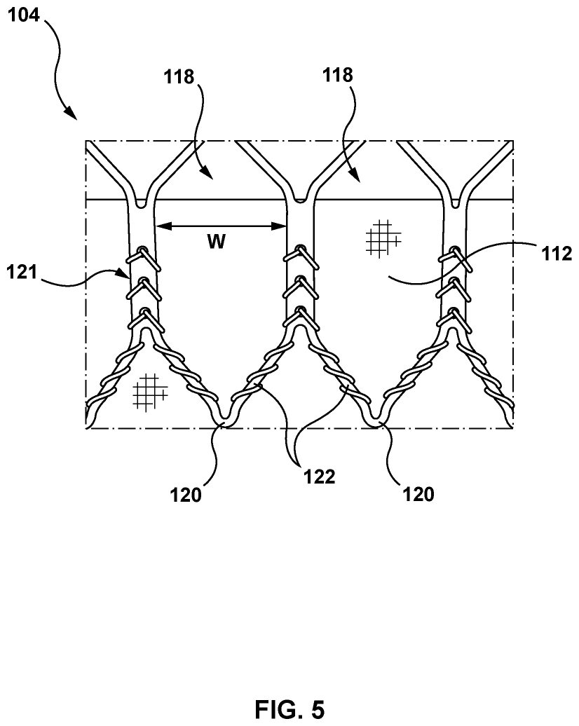

Referring to FIG. 2 as well as FIG. 5, the structure of the valve support 104 will now be described in more detail. FIG. 5 is an enlarged side view of a plurality of side openings 118 of the valve support 104. The valve support 104 includes a plurality of crowns 120 and a plurality of struts 122 with each crown 120 being formed between a pair of opposing struts 122. Each crown 120 is a curved segment or bend extending between opposing struts 122. The valve support 104 is tubular, with the plurality of side openings 118 being defined by edges of the plurality of crowns 120 and the plurality of struts 122. In an embodiment, the plurality of side openings 118 may be substantially diamond-shaped. The valve support 104 includes a plurality of nodes 121. A node 121 is defined as a region where two crowns of the plurality of crowns 120 within the valve support 104 meet or connect. As best shown in FIG. 5, in this embodiment, the skirt 112 is attached to an inner surface of the valve support 104 around a circumference thereof. The skirt 112 lines the inner surface of the valve support 104 and spans across or extends over each side opening 118 of the plurality of side openings 118. Notably, as shown in FIG. 5, it is not required that the skirt 112 extend over the full opening of each side opening 118. Rather, the skirt 112 may span or cover only a portion of the side opening 118 as shown in FIG. 5.

A series of endmost inflow crowns 120A are formed at the inflow end 101 of the valve support 104, and a series of endmost outflow crowns 120B are formed at the outflow end 103 of the valve support 104. In an embodiment, the inflow end 101 of the valve support 104 has a total of nine endmost inflow crowns 120A around a circumference thereof. The inflow end 101 of the valve support 104 includes a row of side openings 118 around a circumference thereof, and the row has a total of nine side openings 118. Further, outflow end 103 of the valve support 104 has a total of nine endmost inflow crowns 120B around a circumference thereof. The outflow end 103 of the valve support 104 includes a row of side openings 118 around a circumference thereof, and the row has a total of nine side openings 118. In another embodiment hereof (not shown), each of the inflow end 101 and the outflow end 103 of the valve support 104 has between six and nine endmost inflow crowns 120A, 120B around a circumference thereof and includes the row of side openings 118 around a circumference thereof that includes between six and nine side openings 118.

A width W of the side openings 118 is relatively wider as compared to other stents or frames known in the art, thereby resulting a relatively lower total of side openings 118 around a circumference of the valve support 104. In an embodiment, width W is between 1/24 and ⅙ of the circumference of the valve support 104, or stated another way, between 4% and 16% of the circumference of the valve support 104. By increasing the width of the side openings 118, a lesser amount of material is required for the valve support 104 such that a lower profile may be achieved when the valve support 104 is crimped into a radially compressed configuration for delivery. More particularly, since the frame 102 includes both the valve support 104 and the anchor element 106, it is a challenge to reduce the profile of the transcatheter valve prosthesis 100 in the crimped or radially compressed configuration. The challenge with reducing the profile is that, in the crimped or radially compressed configuration, the incompressible material of the frame 102 imparts high compressive forces on the soft tissue material of the leaflets 109. Such high compressive forces may alter the integrity of the leaflets 109, thereby impacting the long-term durability of the transcatheter valve prosthesis 100. However, increasing the width W of the side openings 118 provides a reduction of the incompressible material of the frame 102, thereby enabling a lower profile in the crimped or radially compressed configuration.

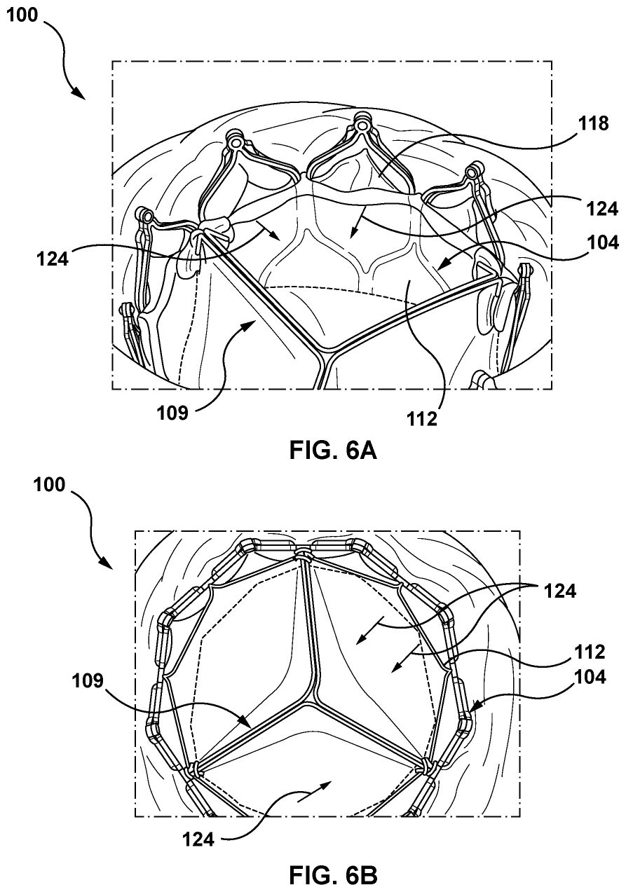

However, reducing the incompressible material of the frame 102 means that the skirt 112 spans a longer distance between nodes 121 or between struts 122. Stated another way, with the width of the side openings 118 being relatively increased as described above, the amount of material of the skirt 112 that spans across the side openings 118 likewise increases and thus a greater amount of material of the skirt 112 is unattached to the valve support 104. Referring now to FIGS. 6A and 6B, when an increased amount of material of the skirt 112 spans across the side openings 118, there is an increased chance of the skirt 112 billowing or moving radially inwards towards the leaflets 109 as indicated by directional arrows 124. The skirt 112 may billow during valve opening and closing in situ, and the leaflets 109 may contact the skirt 112. Such billowing may undesirably result in contact between the skirt 112 and the leaflets 109 of the transcatheter heart valve prosthesis 100. If the leaflets 109 of the transcatheter heart valve prosthesis 100 contact the skirt 112 during opening and closing, such contact may cause early leaflet tissue abrasion as well as early skirt abrasion. Additionally, the greater relative motion between the skirt 112 and the valve support 104 may further induce early skirt abrasion.

Embodiments hereof relate to composite skirts that are configured to limit the radial motion or billowing of the skirt material, thereby minimizing risk of damage to both the skirt and the leaflets 109. For sake of illustration, the composite skirts described herein are incorporated onto the valve support 104, as the structure of the valve support 104 has already been described in detail above. However, as previously stated, the composite skirts described herein may be incorporated onto any stent or frame having a skirt for which it is desirable to limit billowing, and it is not required that the stent or frame include a prosthetic valve component disposed therein.

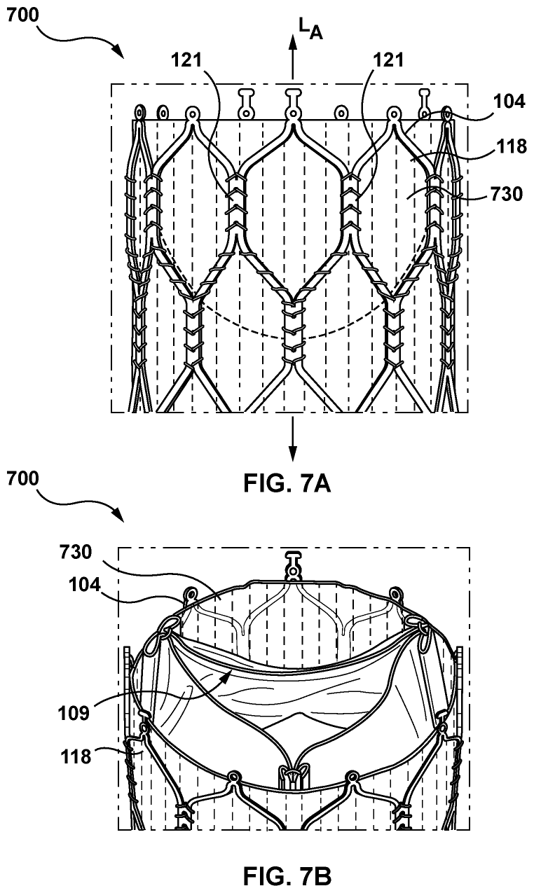

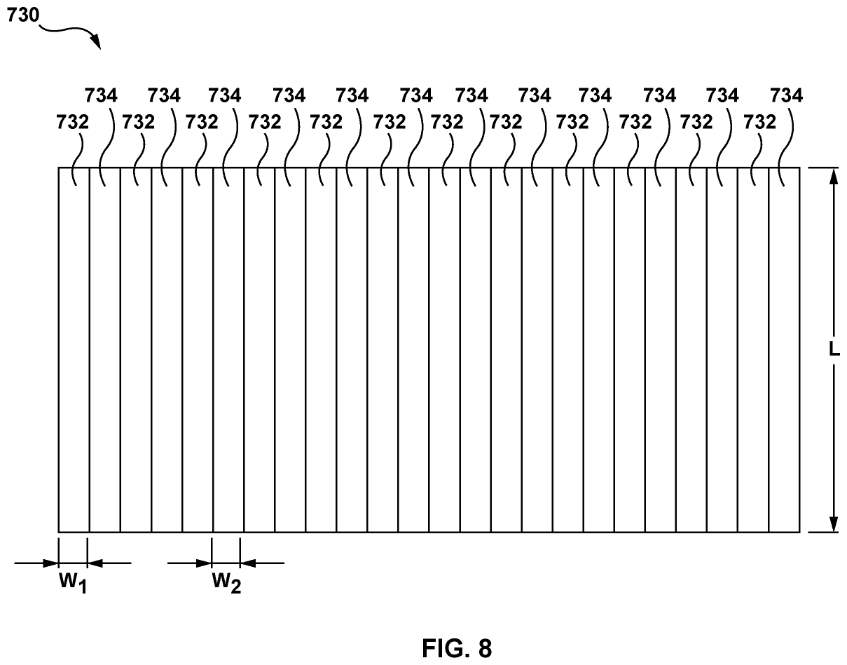

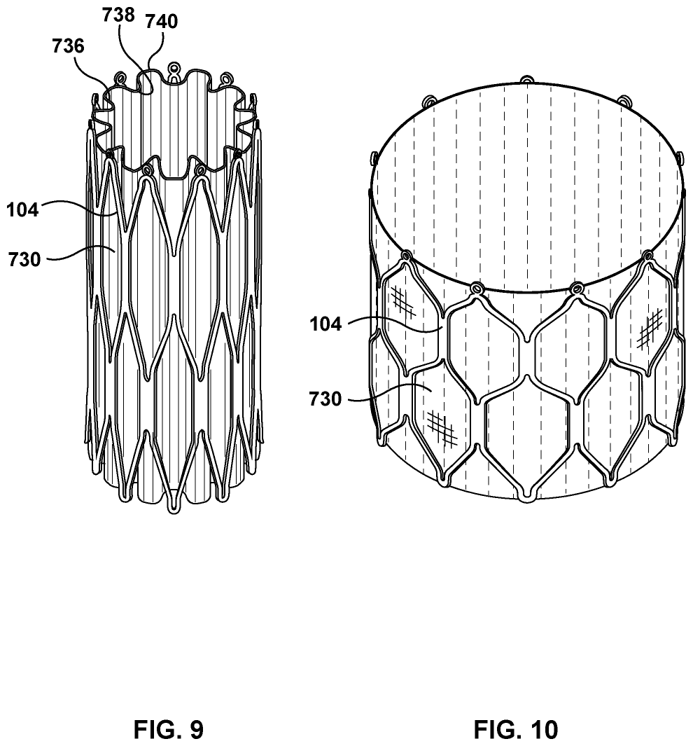

Turning to FIGS. 7A-10, a composite skirt 730 according to an aspect of the disclosure is illustrated on a transcatheter valve prosthesis 700. Transcatheter valve prosthesis 700 includes the valve support 104 as described above, but includes the composite skirt 730 coupled to the valve support 104 rather than the skirt 112. The composite skirt 730 is formed to have direction dependent material properties. As will be explained in more detail herein, the composite skirt 730 includes alternating high and low stiffness segments that are oriented along the main or longitudinal axis LA of the transcatheter valve prosthesis 700. The alternating high and low stiffness segments allow for relatively easy crimping, as the low stiffness segments are configured to fold or collapse radially inward during crimping. In addition, the alternating high and low stiffness segments provide the composite skirt 730 with improved bending stiffness as compared to skirt 112 which has a uniform low-stiffness material, because the high stiffness segments are configured to absorb most of the bending stress applied to the composite skirt, thereby minimizing strain. The improved bending stiffness of composite skirt 730 further manifests in reduced billowing of the skirt material. FIG. 7A and FIG. 7B are side and perspective views, respectively, of the valve support 104 including the composite skirt 730 with the valve support 104 in its radially expanded configuration. FIG. 8 depicts the composite skirt 730 removed from the valve support 104 and laid flat for illustrative purposes only. FIG. 9 is a perspective view of the valve support 104 in its radially compressed configuration, with the prosthetic valve component 108 removed for sake of illustration only, illustrating that the composite skirt 730 has a pleated or folded configuration when the valve support 104 is in its radially compressed configuration. FIG. 10 is a perspective view of the valve support 104 in its radially expanded configuration, with the prosthetic valve component 108 removed for sake of illustration only, illustrating that the composite skirt 730 has an outspread or straightened configuration when the valve support 104 is in its radially expanded configuration.

Particularly, the composite skirt 730 is formed by alternating segments of a first stiffness and a second stiffness that alternate in a circumferential direction around a circumference of the valve support 104. The segments of the first stiffness are referred to herein as first segments 732 and the segments of the second stiffness are referred to herein as second segments 734. The first segments 732 alternate with the second segments 734, or stated another way, each first segment 732 is disposed between a pair of the second segments 734 and each second segment 734 is disposed between a pair of the first segments 732.

Each segment of the alternating first and second segments 732, 734 extends in an axial direction for an entire length L or substantially the entire length L of the composite skirt 730. Stated another way, the cross-section of the composite skirt 730 is the same along an entire length L of the composite skirt 730. As used herein, “substantially the entire length” includes at least 95% of the total or entire length L of the composite skirt. Each segment of the alternating first and second segments 732, 734 is oriented along or disposed parallel to the longitudinal axis LA of the transcatheter valve prosthesis. The first segments 732 of the first stiffness are circumferentially spaced apart at equal intervals around the circumference of the valve support 104, and the second segments 734 of the second stiffness are circumferentially spaced apart at equal intervals around the circumference of the valve support 104.

With particular reference to FIG. 8, each of the first segments 732 have the same size or width W1, and each of the second segments 734 have the same size or width W2. In an embodiment, each segment of the alternating first and second segments 732, 734 are the same size or width such that width W1 is equal to or substantially equal to width W2 although this is not required. Similarly, each of the first segments 732 have the same thickness T1 (not shown) and each of the second segments 734 have the same thickness T2 (not shown). In an embodiment, each segment of the alternating first and second segments 732, 734 have the same thickness such that thickness T1 is equal to or substantially equal to thickness T2 although this is not required. Although FIG. 8 illustrates the composite skirt 730 with twelve first segments 732 and twelve second segments 734, the total number of alternating first and second segments 732, 734 may vary. In an embodiment, the alternating first and second segments 732, 734 include at least five first segments 732 and at least five second segments 734. In an embodiment, the alternating first and second segments 732, 734 include between five and twenty first segments 732 and between five and twenty second segments 734.

The first segments 732 have a first stiffness and the second segments 734 have a second stiffness that is higher than the first stiffness. The second stiffness is at least two times greater or stiffer than the first stiffness. Stated another way, the second segments 734 are at least twice as stiff as the first segments 732. In an embodiment, the first stiffness of the first segments 732 is within an order of magnitude of common cardiovascular fabric materials, such as those described above with respect to the skirt 112. In an embodiment, the composite skirt 730 may be formed from a custom weave of material that includes alternating segments of high and low stiffness properties. In another embodiment, the composite skirt 730 may be formed from a plurality of strips of a first material having the first stiffness and a plurality of strips of a second material having the second stiffness that are attached together in the desired alternating pattern. In such an embodiment, the strips of the first and second materials may be attached to each other via any suitable attachment technique, including adhesive, suture, knitting, or heat (i.e., fusing of fibers). Exemplary materials for the first and second materials include but are not limited to a low-porosity woven fabric, such as Dacron fabric, a knit or woven polyester, or polyester velour fabrics. These and other appropriate cardiovascular fabrics are commercially available from Bard Peripheral Vascular, Inc. of Tempe, Ariz. As described above, and are available in varying densities, thicknesses, and stiffnesses. Thus, the first and second stiffnesses may be achieved by using different materials or may be achieved by using the same material with varying properties, i.e., a different density and/or thickness, to achieve the desired different stiffnesses. In an embodiment, the first and second materials may include a knitted woven PET fabric and the different stiffnesses of the first and second segments may be achieved by varying the amount of warp and fill yarn counts per inch.

The first segments 732 are configured to collapse, bunch, fold, or otherwise move radially inward during crimping of the transcatheter valve prosthesis 700 due to the relatively low stiffness properties thereof. More particularly, the first segments 732 of the composite skirt 730 are configured to fold radially inward when the transcatheter valve prosthesis 700 is in the radially compressed configuration such that the composite skirt 730 has a pleated or folded configuration. Stated another way, the alternating segments 732, 734 of low and high stiffness result in preferential in-folding and out-folding of the unsupported portions of the composite skirt. As used herein, “unsupported portion of the composite skirt” refers to portions or areas of the composite skirt in which a surface of the composite skirt does not directly contact or abut against the frame 104. More particularly, as shown in FIG. 9, within each side opening 118 the unsupported portion of the composite skirt 730 includes a series of alternating pleats 736 defining longitudinal out-folds 740 and longitudinal in-folds 738. Each pleat 736 extends or is oriented in a longitudinal or axial direction, and extends between a longitudinal out-fold 740 and an adjacent longitudinal in-fold 738. Around the circumference of the composite skirt 730, the longitudinal in-folds 738 and longitudinal out-folds 1548 alternate such that a longitudinal out-fold 1548 is disposed between a pair of adjacent longitudinal in-folds 738. Although pleating of the composite skirt 730 may be limited by the stitches that attach the composite skirt 730 to the valve support 104, the overall effect of preferential in-folding and out-folding is still achieved along unsupported portions of the composite skirt 730 to prevent undesired billowing of the skirt material as described herein.

As shown in FIG. 10, when the transcatheter valve prosthesis 700 is in the radially expanded configuration, the composite skirt 730 has an outspread or straightened configuration in which unsupported portions of the composite skirt 730 span straight across or extend over a respective side opening 118 of the valve support 104. The second segments 734 are configured to absorb most of the bending stress applied to the composite skirt 730, thereby minimizing strain and manifesting in reduced billowing of the unsupported portions of the composite skirt 730. Thus, the segment segments 734 having higher stiffness properties provide rigidity to the composite skirt 730 that prevents undesirable billowing of the unsupported portions of the composite skirt 730 when the transcatheter valve prosthesis 700 is in the radially expanded configuration. Stated another way, the second segments 734 of the composite skirt 730 are configured to prevent, restrict, or otherwise mitigate radial movement of the unsupported portions of the composite skirt 730 throughout the cardiac cycle.

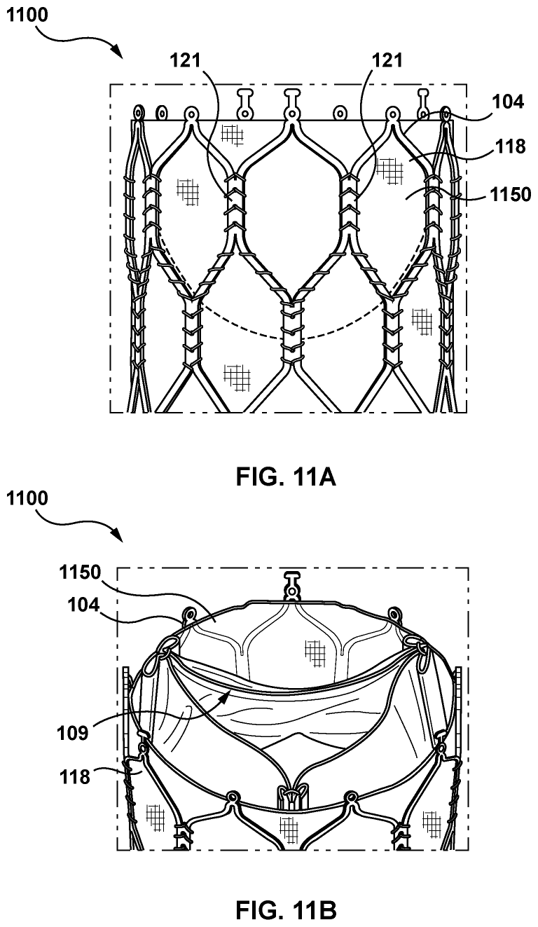

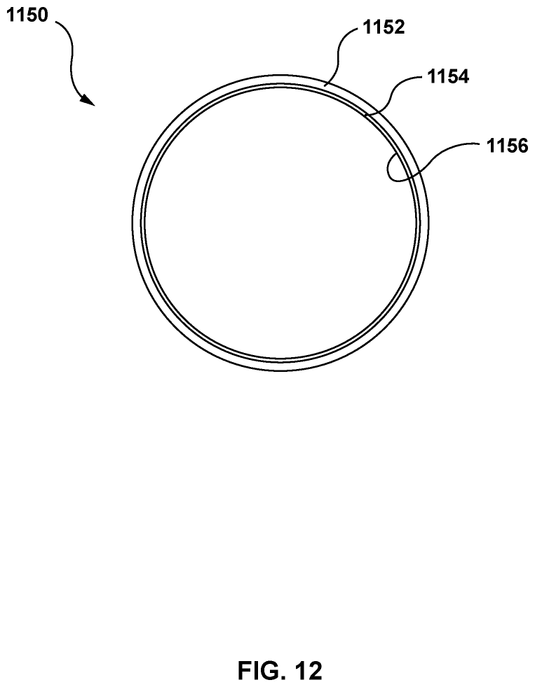

Turning to FIGS. 11A-12, a composite or multi-layer skirt 1150 according to another aspect of the disclosure is illustrated on a transcatheter valve prosthesis 1100. Transcatheter valve prosthesis 1100 includes the valve support 104 as described above, but includes the multi-layer skirt 1150 coupled to the valve support 104 rather than the skirt 112. As will be explained in more detail herein, the multi-layer skirt 1150 is formed from a first or base layer including a uniform low-stiffness fabric and a second or reinforcement layer applied thereto. The fabric layer is very porous and includes loosely woven fibers. The fabric layer is thinner than the skirt 112, which improves or reduces the crimp profile of the transcatheter valve prosthesis 1100 as compared to the transcatheter valve prosthesis 100. The reinforcement layer is a thin polymeric coating that has a higher stiffness than the fabric layer, yet is permitted to elongate to permit crimping of the multi-layer skirt 1150. As such, the reinforcement layer is configured to provide reinforcement for the unsupported portions of the multi-layer skirt 1150 that span straight across or extend over a respective side opening 118 of the valve support 104. As used herein, “unsupported portion of the composite skirt” refers to portions or areas of the composite skirt in which a surface of the composite skirt does not directly contact or abut against the frame 104. FIG. 11A and FIG. 11B are side and perspective views, respectively, of the valve support 104 including the multi-layer skirt 1150 with the valve support 104 in its radially expanded configuration. FIG. 12 depicts a top view of the multi-layer skirt 1150 removed from the valve support 104.

The multi-layer skirt 1150 includes a fabric layer or material 1152 and a reinforcement layer 1154 covering the fabric material 1152 such that the reinforcement layer 1154 defines an inner circumferential surface 1156 of the multi-layer skirt 1150. Although only a top view of the multi-layer skirt 1150 is shown in FIG. 12, each of the fabric layer 1152 and the reinforcement layer 1154 extends in an axial direction for an entire length or substantially the entire length of the multi-layer skirt 1150. Stated another way, the cross-section of the multi-layer skirt 1150 is the same along an entire length of the multi-layer skirt 1150. As used herein, “substantially the entire length” includes at least 95% of the total or entire length of the composite skirt.

The fabric layer or material 1152 is formed by a plurality of loosely woven fibers with a porosity less than 5%. In an embodiment, the fabric layer or material 1152 has a burst strength between 150 N and 300 N, and a suture retention limit between 5 N and 20 N. Exemplary materials for the fabric material include but are not limited to a low-porosity woven fabric, such as Dacron fabric or a knit or woven polyester.

The reinforcement layer 1154 is a polymeric coating. Suitable polymeric coatings include but are not limited to polyurethane (PU), polycarbonate urethane (PCU), or polytetrafluoroethylene (PTFE). In an embodiment, the polymeric coating has a thickness between 2 and 10 um. The polymeric coating or reinforcement layer 1154 is configured to prevent billowing of the multi-layer skirt 1150 when the transcatheter valve prosthesis 1100 is in the radially expanded configuration. The polymeric coating or reinforcement layer 1154 is configured to absorb most of the bending stress applied to the multi-layer skirt 1150, thereby minimizing strain and manifesting in reduced billowing of the unsupported portions of the multi-layer skirt 1150. Thus, the polymeric coating or reinforcement layer 1154 provides rigidity to the multi-layer skirt 1150 that prevents undesirable billowing of the unsupported portions of the multi-layer skirt 1150 when the transcatheter valve prosthesis 1100 is in the radially expanded configuration. Stated another way, the polymeric coating or reinforcement layer 1154 of the multi-layer skirt 1150 is configured to prevent, restrict, or otherwise mitigate radial movement of the unsupported portions of the multi-layer skirt 1150 throughout the cardiac cycle. However, while increasing the stiffness of the multi-layer skirt 1150, the polymeric coating or reinforcement layer 1154 is configured to elongate such that the multi-layer skirt 1150 is also configured to collapse, bunch, fold, or otherwise move radially inward when the transcatheter valve prosthesis 1100 is crimped into its radially compressed configuration.

It will be apparent to one of ordinary skill in the art that the properties of each of the fabric layer or material 1152 and the reinforcement layer 1154 are relative to each other such that the multi-layer skirt 1150 has effective or bulk material properties that perform as described above. For example, as described above, the fabric layer or material 1152 is formed by a plurality of loosely woven fibers with a porosity less than 5% while the polymeric coating has a thickness between 2 and 10 um. The thickness and/or strength of the polymeric coating and the weave density or porosity of the loosely woven fibers are inversely proportional. As the thickness and/or strength of the reinforcement layer 1154 increases, the required weave density or porosity of the fabric layer or material 1152 decreases. Conversely, for relatively higher weave densities or porosities of the fabric layer or material 1152, the thickness of the reinforcement layer 1154 may be relatively decreased.

Further, as stated above, the polymeric coating or reinforcement layer 1154 defines the inner circumferential surface 1156 of the multi-layer skirt 1150. Stated another way, the polymeric coating or reinforcement layer 1154 is disposed on an inner surface of the fabric layer 1152 and thereby forms the inner surface of the multi-layer skirt 1150. Since the reinforcement layer 1154 is a polymeric coating, the inner circumferential surface 1156 of the multi-layer skirt 1150 is smooth and provides a low friction coefficient for reduced abrasion during any leaflet tissue contact. In addition, since the reinforcement layer 1154 is a polymeric coating, the reinforcement layer 1154 provides a sealing function to the multi-layer skirt 1150 such that the multi-layer skirt 1150 creates a one-way fluid passage when attached to the valve support 104.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

1. A prosthesis having a radially expanded configuration and a radially compressed configuration, the prosthesis comprising:
a frame including a plurality of crowns and a plurality of struts with each crown being formed between a pair of opposing struts, wherein a plurality of side openings is defined by the plurality of crowns and the plurality of struts; and
a composite skirt coupled to a surface of the frame, wherein the composite skirt extends over at least one opening of the plurality of side openings of the frame,
wherein the composite skirt is formed by alternating first and second segments of a first stiffness and a second stiffness, respectively, that alternate in a circumferential direction, the second stiffness being higher than the first stiffness, each segment of the alternating first and second segments extending in an axial direction for substantially an entire length of the composite skirt.

2. The prosthesis of claim 1, wherein the first segments of the composite skirt are configured to fold radially inward when the prosthesis is in the radially compressed configuration such that the composite skirt has a pleated configuration when the prosthesis is in the radially compressed configuration.

3. The prosthesis of claim 1, wherein the second segments of the composite skirt are configured to prevent billowing of the composite skirt when the prosthesis is in the radially expanded configuration.

4. The prosthesis of claim 1, wherein the alternating first and second segments include between five and twenty first segments and between five and twenty second segments.

5. The prosthesis of claim 1, wherein each of the first and second segments are the same size.

6. The prosthesis of claim 1, wherein the second stiffness is at least two times greater than the first stiffness.

7. The prosthesis of claim 1, wherein the at least one opening of the plurality of side openings is substantially diamond-shaped and is defined by a total of four struts.

8. The prosthesis of claim 7, wherein each opening of the plurality of side openings is substantially diamond-shaped and is defined by a total of four struts.

9. The prosthesis of claim 8, wherein a perimeter of the frame includes a row of side openings, the row including between six side openings and nine side openings.

10. A prosthesis having a radially expanded configuration and a radially compressed configuration, the prosthesis comprising:
a frame including a plurality of crowns and a plurality of struts with each crown being formed between a pair of opposing struts, wherein a plurality of side openings is defined by the plurality of crowns and the plurality of struts; and
a multi-layer skirt coupled to a surface of the frame, wherein the multi-layer skirt extends over at least one opening of the plurality of side openings of the frame,
wherein the multi-layer skirt is formed by a fabric material and a polymeric coating covering the fabric material such that the polymeric coating defines an inner circumferential surface of the multi-layer skirt, the fabric material having a plurality of fibers with a porosity less than 5% and the polymeric coating having a thickness between 2 and 10 um.

11. The prosthesis of claim 10, wherein the polymeric coating is formed from polyurethane, polycarbonate urethane, or polytetrafluoroethylene.

12. The prosthesis of claim 10, wherein the polymeric coating is configured to prevent billowing of the multi-layer skirt when the prosthesis is in the radially expanded configuration.

13. The prosthesis of claim 10, wherein the at least one opening of the plurality of side openings is substantially diamond-shaped and is defined by a total of four struts.

14. The prosthesis of claim 13, wherein each opening of the plurality of side openings is substantially diamond-shaped and is defined by a total of four struts.

15. The prosthesis of claim 14, wherein a perimeter of the frame includes a row of side openings, the row including between six side openings and nine side openings.

16. The prosthesis of claim 10, wherein the multi-layer skirt is coupled to an inner surface or an outer surface of the frame and wherein the multi-layer skirt extends over each opening of the plurality of side openings of the frame.

17. The prosthesis of claim 10, wherein the frame is formed from a self-expanding material.

18. The prosthesis of claim 10, wherein the frame is an inner frame and the prosthesis further comprises an outer frame coupled to the inner frame, the outer frame radially surrounding the inner frame.

19. The prosthesis of claim 10, wherein the prosthesis is a heart valve prosthesis and the prosthesis further comprises a prosthetic valve component disposed within and secured to the frame, the prosthetic valve component being configured to block blood flow in one direction to regulate blood flow through a central lumen of the frame.

20. The prosthesis of claim 19, wherein the heart valve prosthesis is configured for placement within a mitral heart valve or a tricuspid heart valve in situ.
